(19) United States
(12) Patent Application Publication
(10) Pub. No.: US 2024/0407653 A1
(43) Pub. Date: Dec. 12, 2024

(54) METHODS, SYSTEMS, AND COMPUTER READABLE MEDIA FOR DETECTING NEUROLOGICAL AND/OR PHYSICAL CONDITIONS

(71) Applicant: ARIZONA BOARD OF REGENTS ON BEHALF OF ARIZONA STATE UNIVERSITY, Scottsdale, AZ (US)

(72) Inventor: Thurmon Lockhart, Mesa, AZ (US)

(73) Assignee: ARIZONA BOARD OF REGENTS ON BEHALF OF ARIZONA STATE UNIVERSITY, Scottsdale, AZ (US)

(21) Appl. No.: 18/700,372

(22) PCT Filed: Sep. 30, 2022
(86) PCT No.: PCT/US2022/077445

§ 371 Date: Apr. 11, 2024

Related U.S. Application Data
(60) Provisional application No. 63/256,293, filed on Oct. 15, 2021.

(51) Int. Cl. A61B 5/0205; A61B 5/00; A61B 5/024

(52) U.S. Cl. CPC A61B 5/0205; A61B 5/02438; A61B 5/4082

(57) ABSTRACT

Provided herein are methods of detecting a neurological and/or physical condition in a subject. The methods include receiving physical intensity measures and/or stability measures from the subject to produce a subject data set. The methods also include applying a computational model of temporal and spatial data indicative of the neurological and/or physical condition to the subject data set to identify a substantial match between at least a subset of the subject data set and the computational model of temporal and spatial data. Additional methods as well as related systems and computer readable media are also provided.

### CROSS-REFERENCE TO RELATED APPLICATIONS

This application claims priority to U.S. Provisional Patent Application Ser. No. 63/256,293, filed Oct. 15, 2021, the disclosure of which is incorporated herein by reference.

### BACKGROUND

Nearly four million sports-related concussions are estimated to occur annually. Many concussions go undetected and accordingly, untreated. Yet, even moderate head impacts may cause a traumatic brain injury (TBI), which is a serious and potentially deadly condition.

A variety of concussion assessment systems are currently available. Many of these, however, are not well utilized due to the nature of the associated tests. Some tests, for example, utilize isolation-based assessment conditions that remove athletes from the field of play and are often highly biased. Many sideline concussion assessment systems are also unable to repeat the baseline conditions or measures and fail to detect the effects of concussion on stability. Given that athletes are typically engaged in highly demanding tasks prior to the concussion event, this tends to further bias the stability measures of sway. Moreover, current balance assessment systems generally do not incorporate heart rate (HR) measures as part of their algorithms and accordingly, tend to exhibit bias effects associated with such physical intensities.

Therefore, there is a need for methods, and related aspects, of detecting neurological and/or physical conditions, such as concussion that factor in the effects of physical intensities on balance or sway complexities.

### SUMMARY

The present disclosure relates, in certain aspects, to methods of detecting a neurological and/or physical condition in a subject. These and other aspects will be apparent upon a complete review of the present disclosure, including the accompanying figures.

In one aspect, the present disclosure provides a method of detecting a neurological and/or physical condition in a subject using a computer. The method includes receiving, by the computer, one or more physical intensity measures and/or one or more stability measures from the subject to produce a subject data set. The method also includes applying, by the computer, a computational model of temporal and spatial data indicative of the neurological and/or physical condition to the subject data set to identify a substantial match between at least a subset of the subject data set and the computational model of temporal and spatial data, thereby detecting the neurological and/or physical condition in the subject using the computer. In some embodiments, the method includes receiving the physical intensity measures and/or the stability measures from at least one sensor within communication of at least one target location of the subject. In some embodiments, a wearable device worn by the subject comprises that sensor.

In some embodiments, the physical intensity measures comprise a heart rate intensity measure, a heart rate variability measure, a heart rate interval measure, cardiac stability index (CSI), and/or an electrocardiogram (ECG) measure. In some embodiments, the stability measures comprise a postural stability measure, a gait stability index (GSI), and/or a linear sway measure. In some embodiments, the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure. In some embodiments, the neurological and/or physical condition comprises a concussion, mental fatigue, physical fatigue, bodily injury, traumatic brain injury, and/or frailty.

In some embodiments, the receiving and applying steps are performed in substantially real-time. In some embodiments, the method includes repeating the receiving and applying steps at multiple time points. In some embodiments, the method includes adjusting one or more baseline measures in the subject data set. In some embodiments, the method includes using one or more elements of Floquet theory to generate the computational model of temporal and spatial data indicative of the neurological and/or physical condition.

In one aspect, the present disclosure provides a system that includes a sensor within communication of at least one target location of a subject, which sensor is configured to sense one or more physical intensity measures and/or one or more stability measures from the subject. The system also includes at least one controller operably connected to the sensor, which controller comprises, or is capable of accessing, computer readable media comprising non-transitory computer executable instructions which, when executed by at least one electronic processor, perform at least: receiving, via the sensor, the physical intensity measures and/or the stability measures from the subject to produce a subject data set; and applying a computational model of temporal and spatial data indicative of a neurological and/or physical condition to the subject data set to identify a substantial match between at least a subset of the subject data set and the computational model of temporal and spatial data to detect the neurological and/or physical condition in the subject.

In one aspect, the present disclosure provides a computer readable media comprising non-transitory computer executable instruction which, when executed by an electronic processor perform at least: receiving one or more physical intensity measures and/or one or more stability measures from a subject to produce a subject data set; and applying a computational model of temporal and spatial data indicative of a neurological and/or physical condition to the subject data set to identify a substantial match between at least a subset of the subject data set and the computational model of temporal and spatial data to detect the neurological and/or physical condition in the subject.

In some embodiments of the system or computer readable media disclosed herein, the physical intensity measures comprise a heart rate intensity measure, a heart rate variability measure, a heart rate interval measure, cardiac stability index (CSI), and/or an electrocardiogram (ECG) measure. In some embodiments of the system or computer readable media disclosed herein, the stability measures comprise a postural stability measure, a gait stability index (GSI), and/or a linear sway measure. In some embodiments of the system or computer readable media disclosed herein, the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure. In some embodiments of the system or computer readable media disclosed herein, the neurological and/or physical condition comprises a concussion, mental fatigue, physical fatigue, bodily injury, traumatic brain injury, and/or frailty.

In some embodiments, the system or computer readable media disclosed herein comprise receiving the physical intensity measures and/or the stability measures from at least one sensor within communication of at least one target location of the subject. In some embodiments of the system or computer readable media disclosed herein, a wearable device worn by the subject comprises that sensor. In some embodiments of the system or computer readable media disclosed herein, the receiving and applying steps are performed in substantially real-time. In some embodiments, the system or computer readable media disclosed herein comprise repeating the receiving and applying steps at multiple time points. In some embodiments, the system or computer readable media disclosed herein comprise adjusting one or more baseline measures in the subject data set. In some embodiments, the system or computer readable media disclosed herein comprise using one or more elements of Floquet theory to generate the computational model of temporal and spatial data indicative of the neurological and/or physical condition.

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of detecting a neurological and/or physical condition in a subject using a computer according to some aspects disclosed herein.

FIG. 2 is a schematic diagram of an exemplary system suitable for use with some aspects disclosed herein.

FIG. 3A is a plot of data showing the effect of different heart rate (HR) dynamics (y-axis represents ECG_Max Floquet LS Means, x-axis represents Session) according to some aspects disclosed herein.

FIG. 3B is a plot of data showing the effect of different heart rate (HR) dynamics (y-axis represents ECG_Mean Floquet LS Means, x-axis represents Session) according to some aspects disclosed herein.

FIG. 4A is a plot of data showing the effect that different stances have on sway area (y-axis represents Sway Area (mm²) LS Means, x-axis represents Stance) according to some aspects disclosed herein.

FIG. 4B is a plot of data showing the effect that different eye conditions have on sway area (y-axis represents Sway Area (mm²) LS Means, x-axis represents Condition) according to some aspects disclosed herein.

FIG. 4C is a plot of data showing the effect that different heart rate (HR) dynamics have on sway area (y-axis represents Sway Area (mm²) LS Means, x-axis represents Session) according to some aspects disclosed herein.

FIG. 5 (panels a-e) schematically illustrates the construction of a Poincare map according to some aspects disclosed herein. (a) Raw acceleration (solid line) and angular velocity (dash line); (b) Phase plot (within one gait cycle); (c) Ensemble average phase plot (within one gait cycle); (d) Section of the phase plots at the time of heel contact; (e) Poincare Map with the geometric average as the steady state.

FIG. 6 is a plot of data showing a comparison of whole body (center of mass (COM)) Floquet multipliers (y-axis represents Floquet multiplier, x-axis represents Gait) according to some aspects disclosed herein.

FIG. 7 is a plot of data showing a comparison of Gait Stability Symmetry Index (GSSI) (y-axis represents GSSI, x-axis represents Gait) according to some aspects disclosed herein.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Communicate: As used herein, “communicate” refers to the direct or indirect transfer or transmission, and/or capability of directly or indirectly transferring or transmitting, something at least from one thing to one or more other things or between or among those things. In some embodiments, for example, a sensor detects detectable signals proximal to a target location of a subject, such that the sensor and the target location communicate with one another.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

Substantial Match: As used herein, “substantial match” means that at least a first value or element is at least approximately equal to at least a second value or element. In certain embodiments, for example, a neurological and/or physical condition is detected in a subject when a data set (e.g., values or elements therein) obtained from the subject is at least approximately equal to a computational model of temporal and spatial data (e.g., values or elements therein) that is indicative of the neurological and/or physical condition.

System: As used herein, “system” in the context of medical or scientific instrumentation refers a group of objects and/or devices that form a network for performing a desired objective.

### DETAILED DESCRIPTION

The methods and related aspects of the present disclosure leverage physical intensities, such as heart rate as a measure of concussion and other neurological health conditions. In some embodiments, a wearable heartrate measuring device is used to continuously perform the assessments disclosed in real time. The approaches disclosed herein overcome many existing concussion assessment systems, which frequently involve player or other subject isolation and tend to be highly biased. The assessment methods and systems disclosed herein can repeatedly measure or otherwise account for baseline conditions to thereby minimize or eliminate those conditions as sources of bias. These and other aspects will be apparent upon a complete review of the present disclosure, including the accompanying figures.

As disclosed herein, the effect of heart rate (HR) measurement has significant effect on linear sway variables. In some embodiments, heart rate measurement can be done using unique algorithms and/or sensors incorporated in a wearable device. In some of these embodiments, these wearable HR assessment methods are applied to detect adverse health conditions, such as concussion, mental fatigue, physical fatigue, bodily injury, traumatic brain injury, and frailty. In some embodiments, Floquet theory is used to model the HR variability, electrocardiogram (ECG) and shows that as the physical intensity increases, the HR intervals remains more uniform. Accordingly, this model can be applied to measure stability and more importantly instability/asymmetrical gait and posture as well as can apply to ECG data. This helps to create a sensor agnostic computational model that can incorporate various forms of temporal and spatial information.

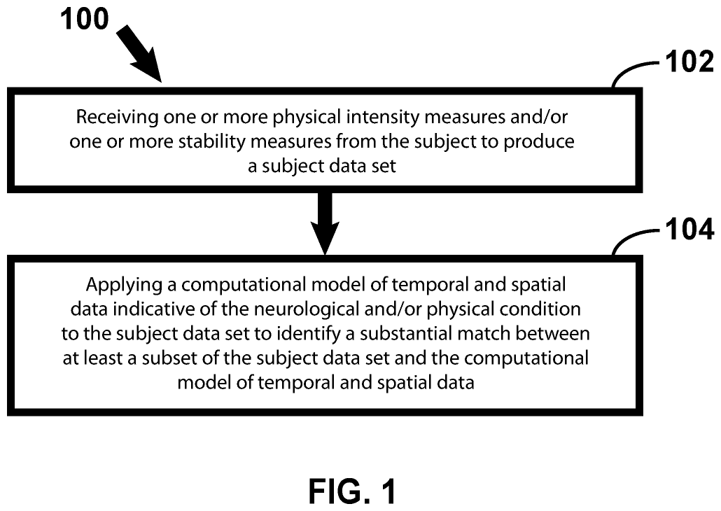

To illustrate, FIG. 1 is a flow chart that schematically shows exemplary method steps of detecting a neurological and/or physical condition in a subject that typically involves the use of a computer. As shown, method 100 includes receiving one or more physical intensity measures and/or one or more stability measures from the subject (e.g., an athlete, a patient, etc.) to produce a subject data set (step 102). Method 100 also includes applying a computational model of temporal and spatial data indicative of the neurological and/or physical condition to the subject data set to identify a substantial match between at least a subset of the subject data set and the computational model of temporal and spatial data to thereby detect the neurological and/or physical condition in the subject (step 104). In some embodiments, method 100 includes receiving the physical intensity measures and/or the stability measures from at least one sensor within communication of at least one target location (e.g., proximal to the xiphoid area, the wrist, etc.) of the subject. In some embodiments, a wearable device (e.g., an article of clothing, a watch, etc.) worn by the subject comprises that sensor.

In some embodiments, the physical intensity measures comprise a heart rate intensity measure, a heart rate variability measure, a heart rate interval measure, cardiac stability index (CSI), and/or an electrocardiogram (ECG) measure. In some embodiments, the stability measures comprise a postural stability measure, a gait stability index (GSI), and/or a linear sway measure. In some embodiments, the stability measures comprise one or more parameters selected from, for example, an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure, among other parameters. In some embodiments, the neurological and/or physical condition comprises a concussion, mental fatigue, physical fatigue, bodily injury, traumatic brain injury, and/or frailty.

In some embodiments, the receiving and applying steps of method 100 are performed in substantially real-time. In some embodiments, method 100 includes repeating the receiving and applying steps at multiple time points. In some embodiments, method 100 includes adjusting, or otherwise accounting for, one or more baseline measures in the subject data set. In some embodiments, method 100 includes using one or more elements of Floquet theory to generate the computational model of temporal and spatial data indicative of the neurological and/or physical condition.

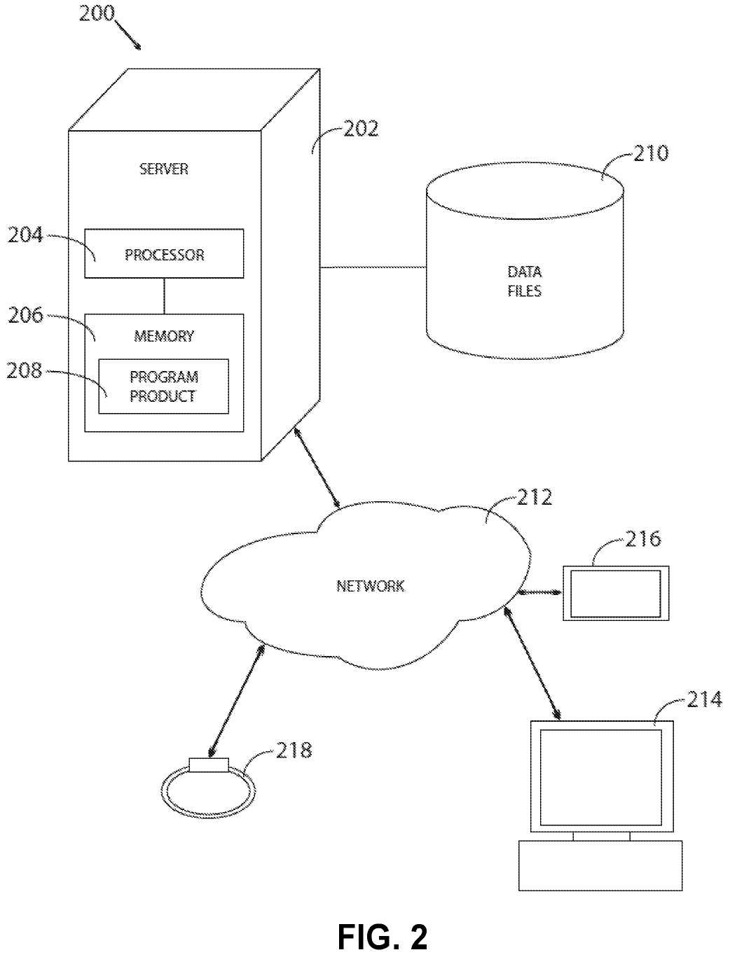

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving subject data sets, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for detecting a neurological and/or physical condition in a subject as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to temporal and spatial data, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to receive the physical intensity measures and/or the stability measures from the subject via sensor 218 (e.g., included as part of a smart watch or other wearable device) to produce a subject data set, to be display data or diagnostic information (e.g., via communication devices 214, 216 or the like) and/or receive information from other system components and/or from a system user (e.g., via communication devices 214, 216, or the like).

In some aspects, program product 208 includes non-transitory computer-executable instructions which, when executed by electronic processor 204, perform at least: receiving one or more physical intensity measures and/or one or more stability measures from a subject (e.g., via sensor 218) to produce a subject data set, and applying a computational model of temporal and spatial data indicative of a neurological and/or physical condition to the subject data set to identify a substantial match between at least a subset of the subject data set and the computational model of temporal and spatial data to detect the neurological and/or physical condition in the subject. Other exemplary executable instructions that are optionally performed are described further herein.

### EXAMPLES

### Example 1: Fractal Rhythm of Heart-Effects of Physical Intensities on Balance Related Concussion Measure

### Introduction

The objective of this example was to determine the association between heart rate intensity and postural stability. The example validated a wearable heartrate measurement method to identify antagonistic health conditions such as concussion, fatigue, and frailty. The example also validated the different stage of physical intensities (i.e., HR) on postural stability complexities that can be successfully used in the field for measuring concussion.

Currently, various concussion assessment systems are used, however, it is not adequately applied due to the structure of the test. A demand associated with a sideline concussion assessment system is capability of repeating the baseline condition measures. This is difficult to measure because players are generally involved in highly demanding on-field tasks prior to the concussion event that may bias stability measures. Moreover, numerous balance assessment systems do not include heart rate into their algorithms and may exhibit bias effects associated with physical intensities.

### Methods

Devices: Polar H10 Heart Rate Sensor that included a belt with the sensor that was positioned on a subject's xiphoid area, GRAIL (Gait Real Time Interactive Laboratory) system for data assessment, and an iPhone mobile telecommunications device having an iPhone mobile application that was used for postural stability data collection. The iPhone device was positioned near the lower back of the subject.

Age-Predicted Maximal Heart Rate Formula: [208−(0.7×Age)×N %].

Adjusted BRUCE protocol method: (1) 30 second push up for each session, and (2) two-minute walking/jogging session.

Sessions: Initial Resting Heart Rate (b), 20% (S1), 40% (S2), 60% (S3), 80% (S4) of Submaximal Targeted Heart Rate, Final Resting Heart Rate (z).

Postural Stability Testing (eyes open (EO)/eyes closed (EC)/tandem stance (TS) (30 sec)).

Subject anthropometric data is shown in Table 1.

TABLE 1

| | Young Adults |
|---|---|
| Age (years) | 21.33 ± 1.53 |
| Height (cm) | 168.93 ± 4.53 |
| Weight (kg) | 70.41 ± 8.88 |
| BMI (kg/m²) | 24.63 ± 1.84 |

Subjects performed postural stability assessment at the Initial resting heart rate, 20%, 40%, 60%, 80%, and final resting heart rate.

Prior to measuring the postural stability, subjects performed 30 second push up in time with metronome 20, 25, 30, and 35 BPM (beat per minute).

Two minutes duration of walking/jogging speed of 0.7, 1.1, 1.5, and 1.8 m/s and inclination of 10%, 12%, 14%, and 16%.

Postural stability (EO, EC, TS) was measured immediately after each session.

### Results

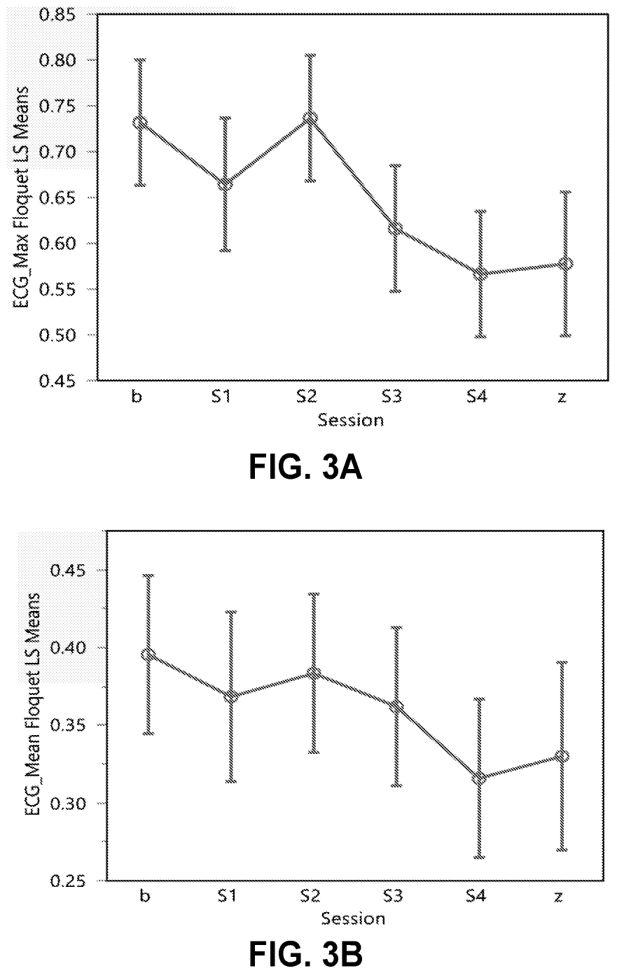

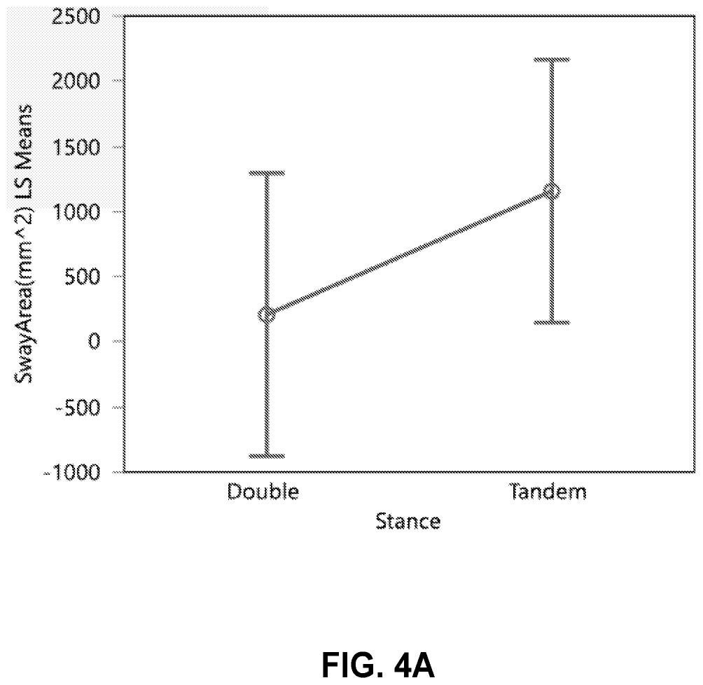

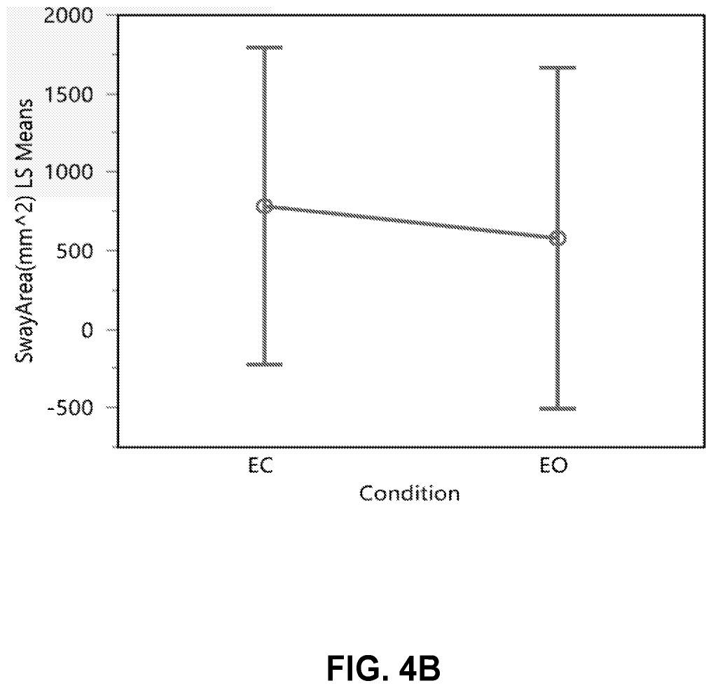

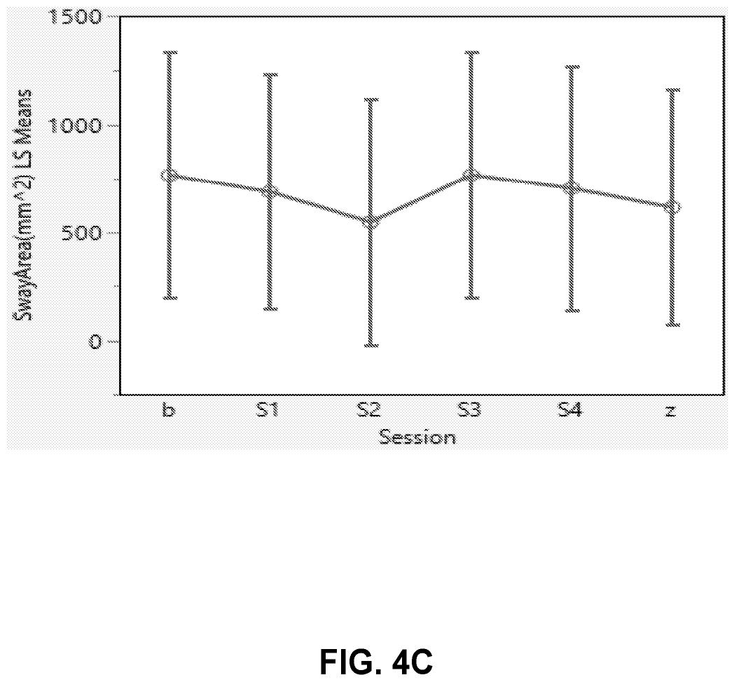

The effect of different heart rate (HR) dynamics is shown in FIGS. 3A and B. The sway area of different stance, eye condition, and HR dynamics is shown in FIGS. 4A-C, respectively. A comparison of iPhone versus force plate sway parameters in shown in Table 2.

TABLE 2

| | | iPhone | | Force Plate | |
|---|---|---|---|---|---|
| | | Double | Tandem | Double | Tandem |
| Confidence | Sway AP (cm) | 9.19 ± 2.41 | 14.55 ± 2.42 | 1.17 ± 0.25 | 4.63 ± 0.25 |
| Score (Max | Sway ML (cm) | 2.92 ± 1.03 | 9.54 ± 1.04 | 3.23 ± 0.33 | 4.03 ± 0.34 |
| 100%) | Sway Path (cm) | 131.08 ± 40.80 | 408.19 ± 40.97 | 113.77 ± 48.67 | 230.87 ± 48.82 |
| | Sway Velocity | 4.68 ± 1.46 | 14.58 ± 1.46 | 3.81 ± 1.63 | 7.70 ± 1.64 |
| | (cm/s) | | | | |
| | Sway Area (cm²) | 15.78 ± 18.79 | 82.20 ± 18.86 | 2.10 ± 1.40 | 11.55 ± 1.41 |
| | RMS AP | 1.92 ± 0.61 | 2.86 ± 0.61 | 2.07 ± 0.57 | 8.67 ± 0.57 |
| | Sample Entropy | 0.52 ± 0.008 | 0.57 ± 0.008 | 1.99 ± 0.004 | 2.01 ± 0.004 |
| | AP | | | | |
| | Sample Entropy | 0.57 ± 0.004 | 0.59 ± 0.004 | 1.98 ± 0.01 | 2.00 ± 0.01 |
| | ML | | | | |

### Conclusions

Floquet Theory was used to model the HR variability, electrocardiogram (ECG) and shown that as the physical intensity increases, the HR intervals remained more uniform. This model can be applied to measure stability and more importantly instability/asymmetrical gait and posture as well as can apply to ECG data. Creating a sensor agnostic computational model that can incorporate various temporal and spatial information. Force plates and iPhone measures were further validated using the factorial design. Thus, a side-line concussion system using iPhone is possible given that the baseline measures are established.

### Example 2: Health (Gait and Posture, and Cardiac) Stability Index

### Orbital Dynamic Stability and GSI Test

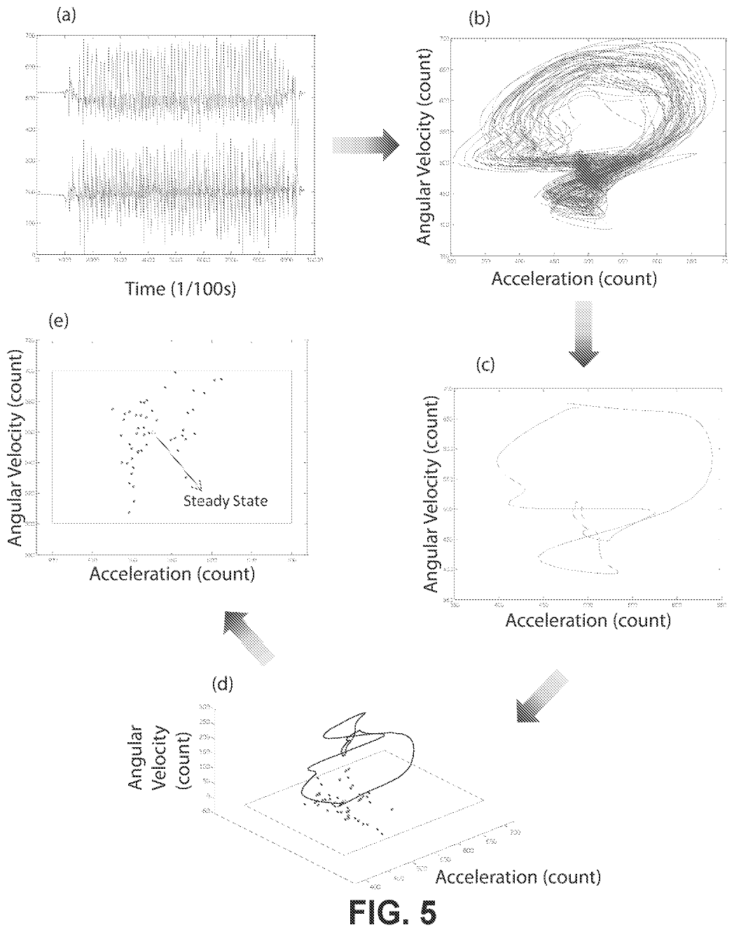

Orbital dynamic stability, as quantified by Floquet multiplier, can be computed using published algorithm in the literature. In this particular case, orbital dynamic stability can be estimated through Poincare analyses of the kinematic dispersion during walking. Vertical acceleration (a) and sagittal angular velocity (w) can be recorded using Inertial Measurement Units (IMUs) from three locations: lower back, left knee, and right knee (FIG. 5a). At each location, these measurements formed into a 2 element state-vector x=(α, ω) (FIG. 5b, c). Heel contacts can be estimated using the characteristics of vertical acceleration at the low back. Gait cycles can further be identified as the period between every other heel contacts. The state-vector of 50 gait cycles can be extracted at each location, and transformed into a state vector of 5000 data points with each gait cycle normalized into 100 points.

The stability control at each instant of the gait cycle can be represented as a nonlinear map of the state-vector x at gait cycle i to the state vector at gait cycle i+1, as illustrated below:

xᵢ₊₁=ƒ(xᵢ).

In this case, the transformation function ƒ( ) is a 2×1 nonlinear representation of movement and describes how the movement kinematics is changed within the time period between the corresponding gait events at gait cycle i and i+1. This is a Poincare section (FIGS. 5d and e) of the walking dynamics system (similarly, heart rate dynamics can be ascertained using this method-presented after the gait example). As each gait cycle is normalized into 100 points, one-hundred Poincare sections will be constructed in this particular case. Through Taylor series expansion of the above equation, the control of a small disturbance Δx of the state-vector x at one instant within gait cycle i can be represented as

$$Δ {x}_{i + 1} = ∇ f ( {x}_{i} ) \cdot Δ {x}_{i} \qquad where Δ {x}_{i + 1} = {x}_{i + 1} - {x}_{i}$$

∇ƒ(xᵢ) is the nonlinear gradient of the transformation function ƒ( ) about the state-vector xᵢ. Given the disturbance experienced during cyclic walking is small, this gradient can be represented as a 2×2 Jacobian matrix J. The disturbance vector at a given instant of each gait cycle i can be computed from the measured state-vector using the following equation

$$Δ {x}_{i} = {x}_{i} - {x}^{*} \qquad where {x}^{*} = 1 / [ n \cdot {\sum }_{j = 1}^{n} {x}_{j} ]$$

x* denotes the equilibrium point of the corresponding Poincare map, and can be estimated as the geometric mean of the state-vector xᵢ in that Poincare map. n is 50 in this case.

Δxᵢ from all the 50 gait cycles can be assembled into a 2 by n−1 (i.e., 2×49) matrix ΔXⱼ, with j denoting j^{th} gait cycle. The transformation equation can be expressed as

$$Δ {X}_{j + 1} = J \cdot Δ {X}_{j}$$

The Jacobian matrix J can then be computed by performing a linear least-square fit of the above equation. With the 2×2 Jacobian matrix, two eigenvalues can be obtained with the maximum eigenvalue (i.e., Floquet multiplier, λ) representing the orbital dynamic stability. The Floquet multipliers usually range from 0 to 1 for repetitive normal waking, with higher value indicating lower orbital stability. Separate Poincare analyses will be performed on the state-vector at each of the three locations. At each location, the above computation will be performed for each instant within the gait cycle. Therefore, three sets of Floquet multipliers with length of 100 will be obtained in this particular case.

### Gait Stability Index (GSI)

Gait Stability Index (GSI) is a novel gait stability measure which is based on orbital dynamic stability. Gait symmetry has been defined as a perfect agreement between the actions of the lower limbs, while others adopt the term “gait symmetry” when no statistical differences are observed on parameters measured bilaterally. It has long been known that gait asymmetry is a direct indicator of various gait pathology, including amputee gait, ACL deficiency, hemiplegic gait, etc. However, the asymmetry in gait dynamic stability has not been addressed and it is hypothesized with GSI, risk of falling and various types of lower limb pathology can be identified.

In this case, GSI is defined the ratio of the difference in Floquet multipliers from left and right side relative to the sum of Floquet multipliers from both sides, as illustrated in the following

$$GSI = \frac{{λ}_{L} - {λ}_{R}}{{λ}_{L} + {λ}_{R}}$$

where λ_{L} and λ_{R} indicate the Floquet multipliers from the left and right side of the lower limb, respectively. The above equation can be reformatted as:

$$GSI = 1 - \frac{2}{\frac{{λ}_{L}}{{λ}_{R}} + 1}$$

Based on this equation, when there is perfect gait stability (λ_{L}=Λ_{R}), GSI equals 0. In the extreme asymmetry case, when λ_{L}>>λ_{R}, GSI approximates to 1. In the other extreme case, when λ_{L}<<Δ_{R}, GSI approximates to −1.

Therefore, GSI ranges from −1 to +1, with 0, −1 and +1 indicating perfect gait stability, extreme asymmetry with lower stability on the right side, extreme asymmetry with lower stability on the left side, respectively.

GSI can be calculated for different various locations of the lower limb. GSI at the hip location can be used as the global indicator can be used as the global indicator of an individual's capability to maintain a symmetrical and stable gait, given that the hip location is close to one's body COM. GSI at the ankle and knee joints can be used as the local indicator of an individual's capability to maintain a symmetrical and stable gait. GSI at the ankle and knee joints can also be used to identify the stability deficit at the ankle and knee joint level.

### Pilot Study Results

The IMMU was attached on the participant according to the following configuration: one sensor on the low back (close to L5/S1), one sensor on the lateral side of the left knee, and one sensor on the lateral side of the right knee. The participants were asked to walk as naturally as possible in a linear hallway (approximately 20 m long). Two normal walking trials and two limping gait trials were collected. The first limping trial was collected from a participant with left knee impairment and the second trial was from a participant with right knee impairment. Each trial contained a dataset corresponding to approximately 10 gait cycles. The Floquet stability and GSI analyses were performed according the methods described in the disclosure.

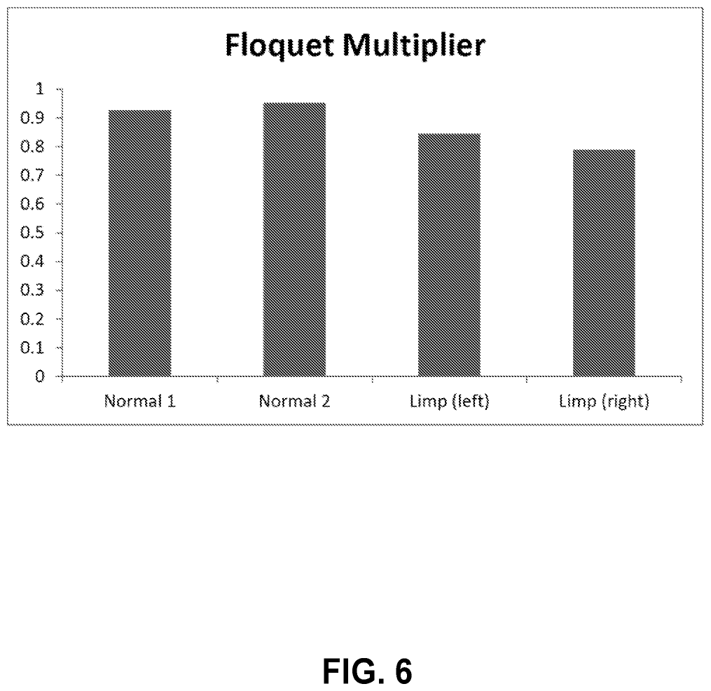

The whole body (center of mass) Floquet stability results were shown in FIG. 6. As illustrated, normal gait trials had slightly higher Floquet multipliers than the limping gait trials. But in general, all the Floquet multipliers were found to be similar, suggesting that both normal gait and limping gait had similar Floquet dynamic stability.

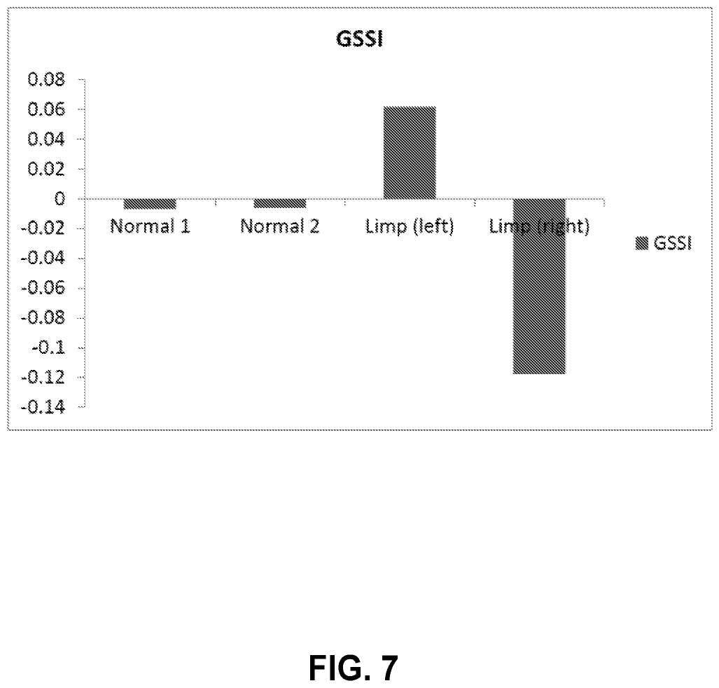

In terms of the dynamic stability symmetry measures, both limping gait trials were found to have considerably higher GSI than the two normal gait trials (FIG. 7). For both normal trials, the GSI were very close to zero, indicating a gait pattern with symmetrical stability. For the limping gait with left knee impairment, the GSI was 0.0617, suggesting a gait stability asymmetry with higher left knee instability. For the limping gait with right knee impairment, the GSI was −0.1176, suggesting a considerable gait stability asymmetry with higher right knee instability.

In conclusion, the pilot study indicated that GSI as a novel stability measure was able to differentiate limping gait from normal gait. Furthermore, the GSI was also able to indicate the side of impairment from the perspective of stability symmetry.

### Opportunity and Technical Problem

Currently kinematics and spatial data can only be effectively captured in a laboratory setting with expensive, cumbersome equipment. There are a few commercially available products that can be used in the health care setting but they do not have the functionality of the devices and system disclosed herein. The data needs to be not only captured in an unobtrusive manner, but also needs to be relayed and analyzed in real-time to give effective, actionable results. The wearable devices and overall monitoring systems disclosed herein form the basis for a commercial system that can be used in a variety of settings and truly improve the health and well-being of many people.

### Pilot Study Results on Heart Rate Stability Index

Floquet theory (more specifically the HRI) was used to model the HR variability (ECG) and shown that as the physical intensity increases, the HR intervals remained more uniform. This model can be applied to measure stability and more importantly instability/asymmetrical gait and posture as well as can apply to ECG data. Creating a sensor agnostic computational model that can incorporate various temporal and spatial information. The study also validated a wearable heartrate measurement method to identify the effects of physical intensities on postural stability. Floquet theory was used to model the HR variability (ECG) and shown that as the physical intensity increases, the HR intervals remained more uniform (FIGS. 3A and B). An objective of this example was to determine the association between heart rate intensity and postural stability. The example validated a wearable heartrate measurement method to identify antagonistic health conditions such as concussion, fatigue, and frailty.

Some further aspects are defined in the following clauses:

Clause 1: A method of detecting a neurological and/or physical condition in a subject using a computer, the method comprising: receiving, by the computer, one or more physical intensity measures and/or one or more stability measures from the subject to produce a subject data set; and, applying, by the computer, a computational model of temporal and spatial data indicative of the neurological and/or physical condition to the subject data set to identify a substantial match between at least a subset of the subject data set and the computational model of temporal and spatial data, thereby detecting the neurological and/or physical condition in the subject using the computer.

Clause 2: The method of Clause 1, wherein the physical intensity measures comprise a heart rate intensity measure, a heart rate variability measure, a heart rate interval measure, cardiac stability index (CSI), and/or an electrocardiogram (ECG) measure.

Clause 3: The method of Clause 1 or Clause 2, wherein the stability measures comprise a postural stability measure, a gait stability index (GSI), and/or a linear sway measure.

Clause 4: The method of any one of the preceding Clauses 1-3, wherein the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure.

Clause 5: The method of any one of the preceding Clauses 1-4, wherein the neurological and/or physical condition comprises a concussion, mental fatigue, physical fatigue, bodily injury, traumatic brain injury, and/or frailty.

Clause 6: The method of any one of the preceding Clauses 1-5, comprising receiving the physical intensity measures and/or the stability measures from at least one sensor within communication of at least one target location of the subject.

Clause 7: The method of any one of the preceding Clauses 1-6, wherein a wearable device worn by the subject comprises that sensor.

Clause 8: The method of any one of the preceding Clauses 1-7, wherein the receiving and applying steps are performed in substantially real-time.

Clause 9: The method of any one of the preceding Clauses 1-8, comprising repeating the receiving and applying steps at multiple time points.

Clause 10: The method of any one of the preceding Clauses 1-9, comprising adjusting one or more baseline measures in the subject data set.

Clause 11: The method of any one of the preceding Clauses 1-10, comprising using one or more elements of Floquet theory to generate the computational model of temporal and spatial data indicative of the neurological and/or physical condition.

Clause 12: A system, comprising: a sensor within communication of at least one target location of a subject, which sensor is configured to sense one or more physical intensity measures and/or one or more stability measures from the subject; and, at least one controller operably connected to the sensor, which controller comprises, or is capable of accessing, computer readable media comprising non-transitory computer executable instructions which, when executed by at least one electronic processor, perform at least: receiving, via the sensor, the physical intensity measures and/or the stability measures from the subject to produce a subject data set; and, applying a computational model of temporal and spatial data indicative of a neurological and/or physical condition to the subject data set to identify a substantial match between at least a subset of the subject data set and the computational model of temporal and spatial data to detect the neurological and/or physical condition in the subject.

Clause 13: The system of Clause 12, wherein the physical intensity measures comprise a heart rate intensity measure, a heart rate variability measure, a heart rate interval measure, cardiac stability index (CSI), and/or an electrocardiogram (ECG) measure.

Clause 14: The system of Clause 12 or Clause 13, wherein the stability measures comprise a postural stability measure, a gait stability index (GSI), and/or a linear sway measure.

Clause 15: The system of any one of the preceding Clauses 12-14, wherein the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure.

Clause 16: The system of any one of the preceding Clauses 12-15, wherein the neurological and/or physical condition comprises a concussion, mental fatigue, physical fatigue, bodily injury, traumatic brain injury, and/or frailty.

Clause 17: The system of any one of the preceding Clauses 12-16, wherein the executable instructions which, when executed by the electronic processor, further perform at least: receiving the physical intensity measures and/or the stability measures from at least one sensor within communication of at least one target location of the subject.

Clause 18: The system of any one of the preceding Clauses 12-17, wherein a wearable device worn by the subject comprises that sensor.

Clause 19: The system of any one of the preceding Clauses 12-18, wherein the receiving and applying steps are performed in substantially real-time.

Clause 20: The system of any one of the preceding Clauses 12-19, wherein the executable instructions which, when executed by the electronic processor, further perform at least: repeating the receiving and applying steps at multiple time points.

Clause 21: The system of any one of the preceding Clauses 12-20, wherein the executable instructions which, when executed by the electronic processor, further perform at least: adjusting one or more baseline measures in the subject data set.

Clause 22: The system of any one of the preceding Clauses 12-21, wherein the executable instructions which, when executed by the electronic processor, further perform at least: using one or more elements of Floquet theory to generate the computational model of temporal and spatial data indicative of the neurological and/or physical condition.

Clause 23: A computer readable media comprising non-transitory computer executable instruction which, when executed by an electronic processor perform at least: receiving one or more physical intensity measures and/or one or more stability measures from a subject to produce a subject data set; and, applying a computational model of temporal and spatial data indicative of a neurological and/or physical condition to the subject data set to identify a substantial match between at least a subset of the subject data set and the computational model of temporal and spatial data to detect the neurological and/or physical condition in the subject.

Clause 24: The computer readable media of Clause 23, wherein the physical intensity measures comprise a heart rate intensity measure, a heart rate variability measure, a heart rate interval measure, cardiac stability index (CSI), and/or an electrocardiogram (ECG) measure.

Clause 25: The computer readable media of Clause 23 or Clause 24, wherein the stability measures comprise a postural stability measure, a gait stability index (GSI), and/or a linear sway measure.

Clause 26: The computer readable media of any one of the preceding Clauses 23-25, wherein the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure.

Clause 27: The computer readable media of any one of the preceding Clauses 23-26, wherein the neurological and/or physical condition comprises a concussion, mental fatigue, physical fatigue, bodily injury, traumatic brain injury, and/or frailty.

Clause 28: The computer readable media of any one of the preceding Clauses 23-27, wherein the executable instructions which, when executed by the electronic processor, further perform at least: receiving the physical intensity measures and/or the stability measures from at least one sensor within communication of at least one target location of the subject.

Clause 29: The computer readable media of any one of the preceding Clauses 23-28, wherein a wearable device worn by the subject comprises that sensor.

Clause 30: The computer readable media of any one of the preceding Clauses 23-29, wherein the receiving and applying steps are performed in substantially real-time.

Clause 31: The computer readable media of any one of the preceding Clauses 23-30, wherein the executable instructions which, when executed by the electronic processor, further perform at least: repeating the receiving and applying steps at multiple time points.

Clause 32: The computer readable media of any one of the preceding Clauses 23-31, wherein the executable instructions which, when executed by the electronic processor, further perform at least: adjusting one or more baseline measures in the subject data set.

Clause 33: The computer readable media of any one of the preceding Clauses 23-32, wherein the executable instructions which, when executed by the electronic processor, further perform at least: using one or more elements of Floquet theory to generate the computational model of temporal and spatial data indicative of the neurological and/or physical condition.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

The invention claimed is:

1. A method of detecting a neurological and/or physical condition in a subject using a computer, the method comprising:
receiving, by the computer, one or more physical intensity measures and/or one or more stability measures from the subject to produce a subject data set; and,
applying, by the computer, a computational model of temporal and spatial data indicative of the neurological and/or physical condition to the subject data set to identify a substantial match between at least a subset of the subject data set and the computational model of temporal and spatial data, thereby detecting the neurological and/or physical condition in the subject using the computer.

2. The method of claim 1, wherein the physical intensity measures comprise a heart rate intensity measure, a heart rate variability measure, a heart rate interval measure, cardiac stability index (CSI), and/or an electrocardiogram (ECG) measure.

3. The method of claim 1, wherein:
the stability measures comprise a postural stability measure, a gait stability index (GSI), and/or a linear sway measure; or,
the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure.

4. (canceled)

5. The method of claim 1, wherein the neurological and/or physical condition comprises a concussion, mental fatigue, physical fatigue, bodily injury, traumatic brain injury, and/or frailty.

6. The method of claim 1, comprising receiving the physical intensity measures and/or the stability measures from at least one sensor within communication of at least one target location of the subject.

7. The method of claim 6, wherein a wearable device worn by the subject comprises that sensor.

8. The method of claim 1, wherein the receiving and applying steps are performed in substantially real-time.

9. The method of claim 1, comprising:
repeating the receiving and applying steps at multiple time points;
adjusting one or more baseline measures in the subject data set; or,
using one or more elements of Floquet theory to generate the computational model of temporal and spatial data indicative of the neurological and/or physical condition.

10. (canceled)

11. (canceled)

12. A system, comprising:
a sensor within communication of at least one target location of a subject, which sensor is configured to sense one or more physical intensity measures and/or one or more stability measures from the subject; and,
at least one controller operably connected to the sensor, which controller comprises, or is capable of accessing, computer readable media comprising non-transitory computer executable instructions which, when executed by at least one electronic processor, perform at least:
receiving, via the sensor, the physical intensity measures and/or the stability measures from the subject to produce a subject data set; and,
applying a computational model of temporal and spatial data indicative of a neurological and/or physical condition to the subject data set to identify a substantial match between at least a subset of the subject data set and the computational model of temporal and spatial data to detect the neurological and/or physical condition in the subject.

13. The system of claim 12, wherein the physical intensity measures comprise a heart rate intensity measure, a heart rate variability measure, a heart rate interval measure, cardiac stability index (CSI), and/or an electrocardiogram (ECG) measure.

14. The system of claim 12, wherein:
the stability measures comprise a postural stability measure, a gait stability index (GSI), and/or a linear sway measure; or,
the stability measures comprise one or more parameters selected from the group consisting of: an eyes open (EO) measure, an eyes closed (EC) measure, a tandem stance (TS) measure, a sway anteroposterior (AP) measure, a sway mediolateral (ML) measure, a sway path measure, a sway velocity measure, a sway area measure, a root mean square AP measure, a sample entropy AP measure, and a sample entropy ML measure.

15. (canceled)

16. The system of claim 12, wherein the neurological and/or physical condition comprises a concussion, mental fatigue, physical fatigue, bodily injury, traumatic brain injury, and/or frailty.

17. The system of claim 12, wherein the executable instructions which, when executed by the electronic processor, further perform at least: receiving the physical intensity measures and/or the stability measures from at least one sensor within communication of at least one target location of the subject.

18. The system of claim 17, wherein a wearable device worn by the subject comprises that sensor.

19. The system of claim 12, wherein the receiving and applying steps are performed in substantially real-time.

20. The system of claim 12, wherein the executable instructions which, when executed by the electronic processor, further perform at least:
repeating the receiving and applying steps at multiple time points;
adjusting one or more baseline measures in the subject data set; or,
using one or more elements of Floquet theory to generate the computational model of temporal and spatial data indicative of the neurological and/or physical condition.

21. (canceled)

22. (canceled)

23. A computer readable media comprising non-transitory computer executable instruction which, when executed by an electronic processor perform at least:
receiving one or more physical intensity measures and/or one or more stability measures from a subject to produce a subject data set; and,
applying a computational model of temporal and spatial data indicative of a neurological and/or physical condition to the subject data set to identify a substantial match between at least a subset of the subject data set and the computational model of temporal and spatial data to detect the neurological and/or physical condition in the subject.

24.-26. (canceled)

27. The computer readable media of claim 23, wherein the neurological and/or physical condition comprises a concussion, mental fatigue, physical fatigue, bodily injury, traumatic brain injury, and/or frailty.

28. The computer readable media of claim 23, wherein the executable instructions which, when executed by the electronic processor, further perform at least: receiving the physical intensity measures and/or the stability measures from at least one sensor within communication of at least one target location of the subject.

29. The computer readable media of claim 28, wherein a wearable device worn by the subject comprises that sensor.

30.-33. (canceled)
